(19) United States
(12) Patent Application Publication
(10) Pub. No.: US 2012/0258150 A1
(43) Pub. Date: Oct. 11, 2012

(54) PARTICLES COMPRISING VOLATILE MATERIALS AND PARTICLE GAS SATURATED SOLUTION PROCESSES FOR MAKING SAME

(76) Inventors: Holly Balasubramanian Rauckhorst, Ft. Thomas, KY (US); Vicenzo D'Acchioli, Francavilla al Mare (IT); Andreas Josef Dreher, Cincinnati, OH (US)

(21) Appl. No.: 13/433,341

(22) Filed: Mar. 29, 2012

Related U.S. Application Data
(60) Provisional application No. 61/474,007, filed on Apr. 11, 2011.

(51) Int. Cl. A61K 8/11; B29B 9/16; A61K 8/02; C11D 17/00; C11D 3/60; A61L 9/012; A61K 9/14; A61K 8/86; A61K 47/30; A61K 47/32; A61K 8/81; A61P 17/00; A61Q 5/02; A61Q 19/00; A61Q 19/10; B32B 27/00; B29B 9/12

(52) U.S. Cl. 424/401; 264/5; 264/7; 510/119; 510/130; 510/276; 510/218; 510/447; 510/108; 510/475; 512/4; 424/400; 514/772; 514/772.4; 428/402.21; 428/407

(57) ABSTRACT

Particles containing a polymer and a volatile material, such as a perfume, and particle gas saturated solution (PGSS) processes for making such particles are provided.

### CROSS REFERENCE TO RELATED APPLICATION

This application claims the benefit of U.S. Provisional Application No. 61/474,007, filed Apr. 11, 2011.

### FIELD OF THE INVENTION

The present invention relates to particles comprising volatile materials and more particularly to particles comprising a perfume and a polymer, and particle gas saturated solution (PGSS) processes for making such particles.

### BACKGROUND OF THE INVENTION

Polymeric materials that contain a volatile material, such as a perfume, are well known in the art. One problem encountered when making particles from such polymeric materials is that they tend to agglomerate rather than remain as discrete particles.

Polymeric materials containing volatile materials, such as perfumes, can be made by encapsulation processes. Encapsulation of volatile materials, such as perfume or other materials, in small capsules (or microcapsules), typically having a diameter less than 1000 microns, is well known. Various types of microcapsules for encapsulating perfumes exist, e.g. polymeric particles, cyclodextrin/perfume inclusion complexes, polysaccharide cellular murices. One type of capsule referred to as a wall or shell capsule comprises a generally spherical hollow shell of insoluble material, typically polymer material, within which the volatile material, for example perfume, is contained. The shell capsules may be prepared using a range of conventional methods known to those skilled in the art for making shell capsules such as coacervation, interfacial polymerization and poly-condensation. The process of coacervation typically involves encapsulation of a generally water-insoluble material by the precipitation of colloidal material(s) onto the surface of droplets of the material. Coacervation may be simple e.g. using one colloid such as gelatin, or complex where two or possibly more colloids of opposite charge, such as gelatin and gum arabic or gelatin and carboxymethyl cellulose, are used under carefully controlled conditions of pH, temperature and concentration.

Interfacial polymerization produces encapsulated shells from the reaction of at least one oil-soluble wall forming material present in the oil phase with at least one water-soluble wall forming material present in the aqueous phase. A polymerization reaction between the two wall-forming materials occurs resulting in the formation of covalent bonds at the interface of the oil and aqueous phases to form the capsule wall. An example of a shell capsule produced by this method is a polyurethane capsule.

Polycondensation involves forming a dispersion or emulsion of a water-insoluble volatile material, for example a perfume, in an aqueous solution of precondensate of polymeric materials under appropriate conditions of agitation to produce capsules of a desired size, and adjusting the reaction conditions to cause condensation of the precondensate by acid catalysis, resulting in the condensate separating from solution and surrounding the dispersed water-insoluble volatile material to produce a coherent film and the desired microcapsules. The shells of the microcapsules are typical made from polymers selected from the group consisting of: urea-formaldehyde, melamine-formaldehyde, phenol-formaldehyde, gelatin, polyurethane, polyamides, cellulose esters including cellulose butyrate, acetate and cellulose nitrate, cellulose ethers like ethyl cellulose, polymethacrylates.

Each of these known processes for making microcapsules and/or particles of polymer and volatile material have their disadvantages, whether it be the materials used, such as formaldehyde, or the conditions under which the processes are run. In addition, due to the conditions under which the known processes are run, the materials suitable for use in such processes are relatively limited.

Accordingly, there is a need for particles that comprise a polymer, such as a copolymer of ethylene with at least another monomer comprising at least a heteroatom, and a volatile material, such as a perfume, that avoids the problems associated with known processes such as broadens the scope of suitable polymers that can be used in the particles compared to particles produced by known processes, and/or particles that comprise a polymer and a volatile material that are made by a PGSS process, and such particles that are suitable for use in various consumer products.

### SUMMARY OF THE INVENTION

The present invention fulfills the need by providing a particle comprising a polymer (and/or a lipophilic agent) and a volatile material and a PGSS process for making such particles.

In one example of the present invention, a particle comprising at least a polymer, such as a copolymer of ethylene with at least another monomer comprising at least a heteroatom, and a volatile material, such as a perfume, is provided.

In still another example of the present invention, a particle comprising a polymer and a volatile material, wherein the particle is produced by a PGSS process, is provided.

In another example of the present invention, a particle comprising a polymer and a volatile material produced by a PGSS process, wherein the particle exhibits novel properties, is provided.

In even another example of the present invention, a process for producing a particle according to the present invention, wherein the process comprises depressurizing a solution comprising a polymer and a volatile material and a highly compressible fluid dissolved in the solution such that a particle comprising the polymer and volatile material is produced, is provided.

In another example of the present invention, a particle comprising a polymer and a volatile material, wherein the particle is produced by a PGSS process and wherein the particle exhibits novel properties, is provided.

Accordingly, the present invention provides particles that comprise a polymer, such as a copolymer of ethylene with at least another monomer comprising at least a heteroatom, and a volatile material, such as a perfume, and such particle produced by a PGSS process.

### BRIEF DESCRIPTION OF THE DRAWINGS

FIG. 1A is a schematic representation of an example of a particle according to the present invention;

FIG. 1B is a cross-sectional view of the particle of FIG. 1A;

FIG. 2A is a schematic representation of an example of a particle according to the present invention;

FIG. 2B is a cross-sectional view of the particle of FIG. 2A;

FIG. 3A is a schematic representation of an example of a particle according to the present invention;

FIG. 3B is a cross-sectional view of the particle of FIG. 3A;

FIG. 4A is a schematic representation of an example of a particle according to the present invention;

FIG. 4B is a cross-sectional view of the particle of FIG. 4A;

FIG. 5A is a schematic representation of an example of a particle according to the present invention;

FIG. 5B is a cross-sectional view of the particle of FIG. 5A; and

FIG. 6 is a schematic representation of a PGSS process for producing particles according to the present invention.

### DETAILED DESCRIPTION OF THE INVENTION

### Definitions

“Particle” as used herein means a composite, multi-component particulate or powder. In one example, the particle may be generally spherical in shape. In another example, the particle exhibits a Morphology Coefficient F of greater than 0.2 and/or greater than 0.4 and/or greater than 0.6 and/or greater than 0.8. In another example, the particle is a solid material produced from a PGSS process.

The particle may exhibit an average particle size of less than 1 mm and/or less than 500 μm and/or less than 250 μm and/or less than 100 μm and/or less than 50 μm and/or less than 30 μm and/or less than 20 μm and/or greater than 1 nm and/or greater than 100 nm and/or greater than 1 μm as measured according to the Particle Size Test Method described herein.

“Average particle size” as used herein for a material, such as a solid additive in accordance with the present invention, is determined according to the Particle Size Test Method described herein. The units for average particle size as used herein are μm.

“Volatile material” as used herein means a material that generates vapors under usage conditions, for example its vapor pressure is at least 0.1 mm Hg at 23° C.±2.2° C. Non-limiting examples of volatile materials include perfumes, flavors, deodorants, insecticides, pheromones, aromas, and repellants.

“Perfume” as used herein means any odoriferous material. In one example, a perfume is a volatile material with a relatively high vapor pressure. In another example, a perfume is a volatile material that exhibits a vapor pressure of at least 0.1 mm Hg to less than atmospheric pressure at 23° C.±2.2° C. The perfumes employed herein will most often be liquid at 23° C.±2.2° C., but also can be solid such as the various camphoraceous perfumes known in the art. A wide variety of chemicals are known for perfumery uses, including materials such as aldehydes, ketones, esters, alcohols, terpenes and the like. Naturally occurring plant and animal oils and exudates comprising complex mixtures of various chemical components are known for use as perfumes, and such materials can be used herein. The perfumes herein can be relatively simple in their composition or can comprise highly sophisticated, complex mixtures of natural and synthetic chemical components, all chosen to provide any desired odor.

“Water-soluble” as used herein with reference to a material, such as a polymer, means a material that exhibits a solubility of at least 5% and/or greater than 10% and/or greater than 30% and/or greater than 50% and/or greater than 75% to 100% by weight in distilled water. Solubility is defined as creation of a single phase from two or more materials at room temperature (23° C.±2.2°).

“Water-insoluble” as used herein with reference to a material, such as a polymer, means a material that exhibits a solubility of less than 5% and/or less than 3% and/or less than 1% by weight in distilled water. Solubility is defined as creation of a single phase from two or more materials at room temperature (23° C.±2.2°).

“Lipophilic agent” as used herein means a water-insoluble material. Even if a material is considered water-soluble as described above, the material may still be a lipophilic agent if the material exhibits a contact angle of greater than 80° and/or greater than 90° and/or greater than 100° and/or greater than 110° and/or greater than 120° as measured according to the Contact Angle Test Method described herein.

“Non-ingestible” as used herein means that a material and/or particle is not suitable and/or intended for ingestion by a human and/or animal. For example, a non-ingestible particle is a particle that is not suitable and/or intended to be swallowed by a human and/or animal.

“Morphology Coefficient F” as used herein is a mathematical characterization of a particle of the present invention, for example a particle produced by a PGSS process. The Morphology Coefficient F of a particle is determined by the following equation:

$$Morphology Coefficient F = 6.9 \times {10}^{- 11} \frac{{( T , Kelvin )}^{4.247}}{{( p , bar )}^{0.043} \times {GTP}^{0.105}}$$

wherein p is the spraying pressure, T is the temperature pre-decompression, and GTP is the gas to particle ratio.

### Particles

The particles of the present invention may comprise one or more polymers and one or more volatile materials. In one example, the particles of the present invention may comprise a polymer, such as a copolymer of ethylene with at least another monomer comprising at least a heteroatom, and a volatile material, such as a perfume.

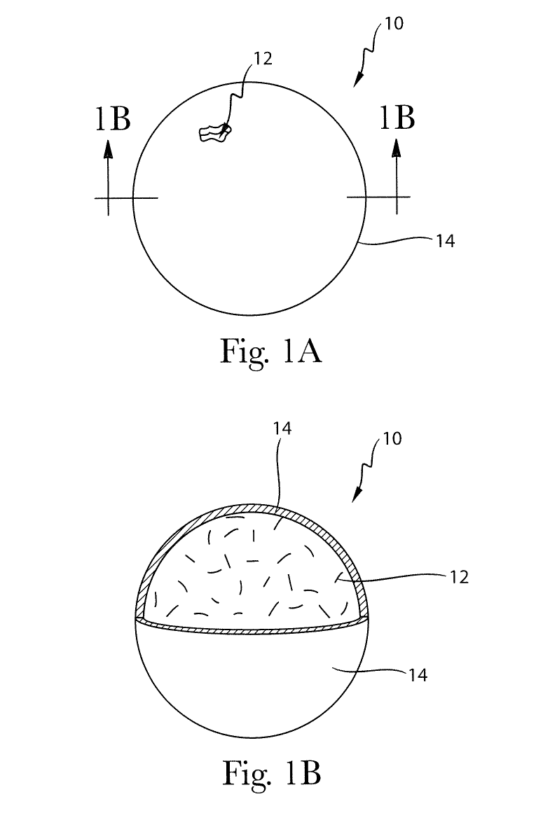

In one example as shown in FIGS. 1A and 1B, a particle 10 of the present invention may comprise a liquid core material 12, such as a volatile material, for example a perfume, encapsulated within a solid shell material 14, such as a polymer, for example a copolymer of ethylene with at least another monomer comprising at least a heteroatom. The solid shell material 14 may be a non-porous shell such that the liquid core material 12 is not permitted to pass through the non-porous shell to the external environment until during use. Alternatively, the solid shell material 14 may be a porous shell such that the liquid core material 12 is capable of passing through the porous shell to the external environment. In another example, the solid shell material 14 may permit the liquid core material 12 to diffuse from the interior of the shell to the exterior of the shell.

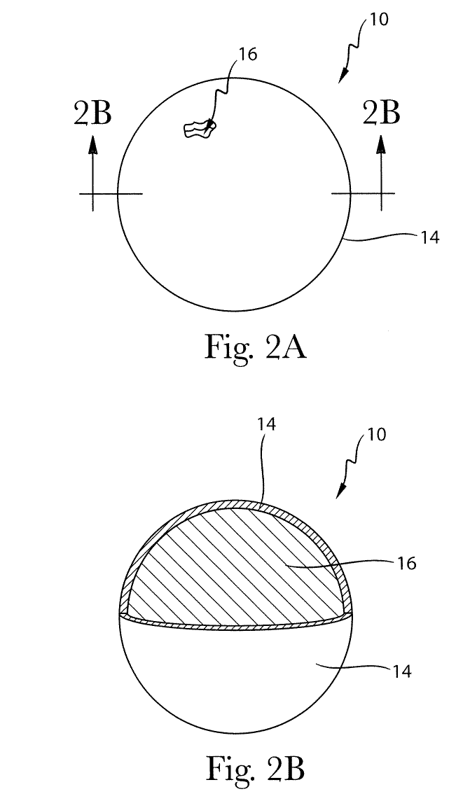

In another example as shown in FIGS. 2A and 2B, a particle 10 of the present invention may comprise a solid core material 16, such as a volatile material, for example a perfume, encapsulated within a solid shell material 14, such as a polymer, for example a copolymer of ethylene with at least another monomer comprising at least a heteroatom. The solid shell material 14 may be a porous or non-porous shell.

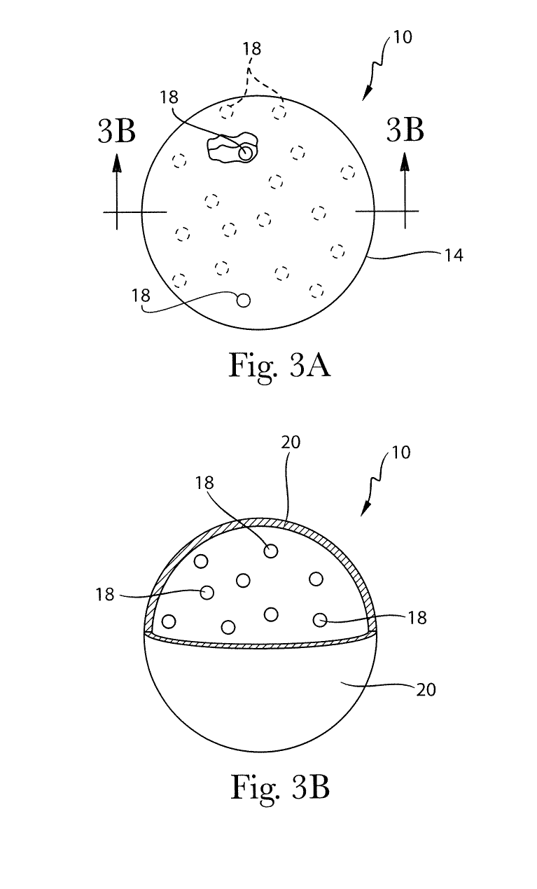

In even another example as shown in FIGS. 3A and 3B, a particle 10 of the present invention may comprise one or more liquid islands 18 of a material, such as a volatile material, for example a perfume, and a solid matrix material 20, such as a polymer, for example a copolymer of ethylene with at least another monomer comprising at least a heteroatom.

In one example, the solid matrix material 20 of the present invention may be a gel, which may be a solid, jelly-like material. In one example, the solid matrix material 20 is a gel comprising a dispersion of solid particles within a liquid in which the solid particles constitute a discontinuous phase and the liquid constitutes a continuous phase

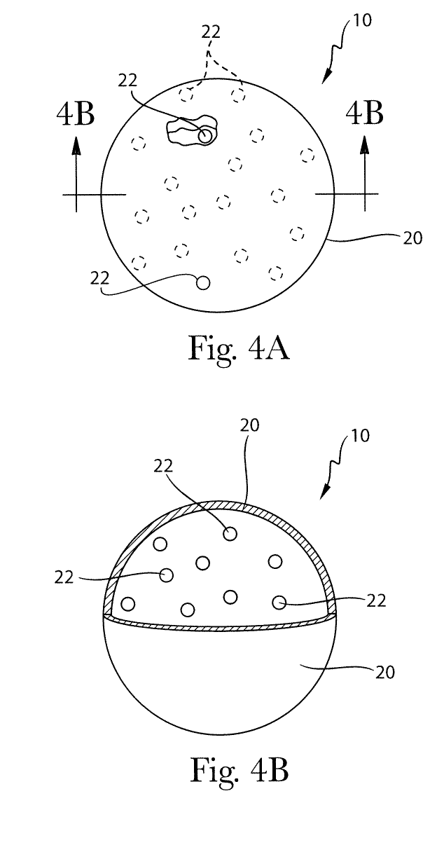

In another example, the solid matrix material 20 of the present invention may be a colloid, where at least one material is microscopically dispersed evenly throughout another material. In even yet another example as shown in FIGS. 4A and 4B, a particle 10 of the present invention may comprise one or more solid islands 22 of a material, such as a hydrophilic material, for example glycerin and a solid matrix material 20, such as a lipophilic material, for example petrolatum.

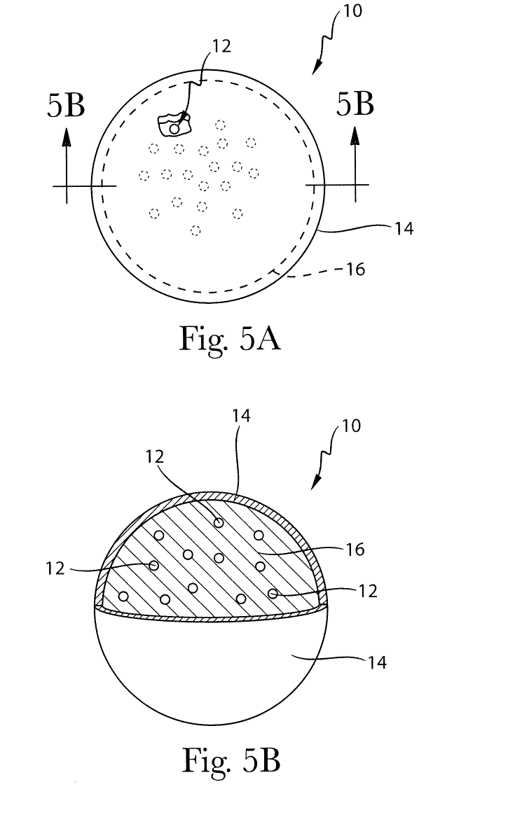

In still yet another example as shown in FIGS. 5A and 5B, a particle 10 of the present invention may comprise a mixture of liquid core material 12 and solid core material 16 dispersed within the liquid core material 12 and a solid shell material 14, which may be porous or non-porous. Alternatively, a solid matrix material may replace the solid shell material in this example.

In addition to the configurations described above of the incompatible materials within the particle, the reverse configurations, such as the polymer being a “core” material and the volatile material being a “shell” material in the various examples shown in FIGS. 1 through 5, are also within the scope of the present invention.

The particle of the present invention may exhibit an average particle size of less than 1 mm and/or less than 500 μm and/or less than 250 μm and/or less than 100 μm and/or greater than 1 μm as measured according to the Particle Size Test Method described herein.

In one example, greater than 80% of a plurality of particles of the present invention exhibit a particle size of between 200 μm and 500 nm as measured according to the Particle Size Test Method described herein.

In another example, the plurality of particles exhibit an average particle size distribution from about 250 μm to 100 nm as measured according to the Particle Size Test Method described herein.

The particle exhibits a Morphology Coefficient F of greater than 0.2 and/or greater than 0.4 and/or greater than 0.6 and/or greater than 0.8.

In one example, the particle of the present invention comprises a weight ratio of volatile material to polymer of greater than 1:10 and/or greater than 1:5 and/or greater than 2:5 and/or greater than 1:2 and/or less than 10:1 and/or less than 5:1 and/or less than 5:2 and/or less than 2:1. In one example, the weight ratio of volatile material to polymer in a particle of the present invention is about 1:1.

In another example, the particle of the present invention may comprise greater than 5% and/or greater than 10% and/or greater than 20% and/or greater than 40% and/or greater than 50% and/or less than 95% and/or less than 90% and/or less than 80% and/or less than 60% by weight of a volatile material and less than 95% and/or less than 90% and/or less than 80% and/or less than 60% and/or less than 50% and/or greater than 5% and/or greater than 10% and/or greater than 20% and/or greater than 40% by weight of a polymer.

In one example, the particle of the present invention comprises from about 5% to about 75% and/or from about 10% to about 50% by weight of the particle of a polymer, such as a copolymer of ethylene with at least another monomer comprising at least a heteroatom; from about 10% to about 60% and/or from about 15% to about 40% by weight of the polymer, of a compatible tackifier, up to 10% by weight of the particle of a plasticizer and/or phase change solvent, and greater than 10% and/or greater than 20% and/or greater than 30% by weight of the particle of a volatile material.

In another example, the volatile material comprises up to 90% by weight of the particle.

In one example, the particles of the present invention comprise one or more volatile materials and one or more polymers. In one example, the volatile material may be a perfume and/or perfume raw material. In yet another example, the particles of the present invention comprise one or more volatile materials, one or more polymers, and one or more tackifiers and/or one or more plasticizers. In even another example, the particles of the present invention comprise one or more volatile materials and one or more lipophilic agents.

In one example, the polymers of the present invention may comprise water-insoluble polymers, for example polymers other than polyethylene glycol and/or other than polysaccharides, such as starch including starch derivatives.

In another example, the particles of the present invention comprise non-ingestible particles.

In one example, a consumer product, for example a consumer product selected from the group consisting of: shampoos, body washes, laundry detergents, dishwashing detergents, anhydrous liquid products, bar soaps, paper products, cosmetics, lotions, skin treating products, and mixtures thereof, may comprise one or more particles of the present invention.

### Volatile Materials

Non-limiting examples of volatile materials suitable for the present invention include perfumes and perfume raw materials. Perfumes are typically composed of many components of different volatility. The present invention, avoiding separation of the components based on their different volatility, allows the sustained delivery of the full perfume bouquet for a long time. In a preferred embodiment of the present invention the volatile material is a perfume which is preferably composed by a plurality of components, more preferably by more than 5 components.

Non-limiting examples of suitable perfumes include woody/earthy bases containing exotic materials such as sandalwood oil, civet, patchouli oil and the like. Other suitable perfumes are for example light, floral fragrances, e.g., rose extract, violet extract and the like. Perfumes can be formulated to provide desirable fruity odors, e.g., lime, lemon, orange and the like. In short, any chemically compatible material that emanates a pleasant or otherwise desirable odor can be used as a perfume in the present invention.

Further examples of suitable perfumes are described more fully in S. Arctander, Perfume and Flavor Chemicals (Aroma Chemicals), Vols. I and II, Montclair, N.J., and the Merck Index, 8th Edition, Merck & Co., Inc. Rahway, N.J.

Non-limiting examples of suitable volatile materials included perfume raw materials. Non-limiting examples of suitable perfume raw materials (including any stereoisomers thereof and any mixtures thereof) are set forth in Table 1.

TABLE 1
Suitable Perfume Raw Materials (“PRMs”)

| Common Name | IUPAC Name (Chemical Name) |
|---|---|
| (+)-D-menthyl | (5-methyl-2-propan-2-ylcyclohexyl) acetate |
| acetate | |
| (I)-citronellal | 3,7-dimethyloct-6-en-1-al |
| 2-phenoxyethyl | 2-(phenoxy)ethyl 2-methylpropanoate |
| isobutyrate | |
| allyl amyl | prop-2-enyl 2-(3-methylbutoxy)acetate |
| glycolate | |
| alicate | 3-methyl-1-isobutylbutyl acetate |
| allyl caproate | prop-2-enyl hexanoate |
| allyl cyclohexyl | prop-2-enyl 3-cyclohexylpropanoate |
| propionate | |
| allyl heptanoate | prop-2-enyl heptanoate |
| alpha damascone | (E)-1-(2,6,6-trimethyl-1-cyclohex-2-enyl)but-2-en-1-one |
| alpha Ionone | (E)-4-(2,6,6-trimethyl-1-cyclohex-2-enyl)but-3-en-2-one |
| alpha | (E)-3-methyl-4-(2,6,6-trimethyl-1-cyclohex-2-enyl)but-3-en-2-one |
| isomethylionone | |
| alpha methyl | 1-(2,6,6-trimethyl-1-cyclohex-2-enyl)pent-1-en-3-one |
| ionone | |
| ambrox | 6,6,9a-trimethyl-1,2,3a,4,5,5a,7,8,9,9b- |
| | decahydronaphtho[2,1-b]furan |
| amyl salicylate | pentyl 2-hydroxybenzoate |
| beta pinene | 7,7-dimethyl-2-methylidene-norbornane |
| beta-damascone | (E)-1-(2,6,6-trimethyl-1-cyclohexenyl)but-2-en-1-one |
| beta-ionone | (E)-4-(2,6,6-trimethyl-1-cyclohexenyl)but-3-en-2-one |
| boisiris | 4-ethoxy-4,8,8-trimethyl-9- |
| | methylidenebicyclo[3.3.1]nonane |
| bornyl acetate | (1,7,7-trimethyl-6-bicyclo[2.2.1]heptanyl) acetate |
| bourgeonal | 3-(4-tert-butylphenyl)propanal |
| cashmeran | 1,1,2,3,3-pentamethyl-2,5,6,7-tetrahydroinden-4-one |
| cassifix | 2-oxabicyclo2.2.2octane,1methyl4(2,2,3trimethylcyclopentyl) |
| cis-3-hexenyl | [(Z)-hex-3-enyl] acetate |
| acetate | |
| cis-3-hexenyl | [(Z)-hex-3-enyl] 2-methylbutanoate |
| methyl butyrate | |
| cis-3-hexenyl | cis-3-hexenyl 2-hydroxybenzoate |
| salicylate | |
| citral | 3,7-dimethylocta-2,6-dienal |
| citronellal | 3,7-dimethyloct-6-en-1-al |
| citronellol | 3,7-dimethyl-6-octen-1-ol |
| citronellyl acetate | 3,7-dimethyloct-6-enyl acetate |
| citronellyl nitrile | 3,7-dimethyloct-6-enenitrile |
| citronellyl | 2-(3,7-dimethyloct-6-enoxy)acetaldehyde |
| oxyacetaldehyde | |
| clarycet | tetrahydro-4-methyl-2-propyl-2h-pyran-4-yl acetate |
| coranol | ethyl 3-phenyloxirane-2-carboxylate |
| cyclabute | hexahydro-4,7-methano-indenyl isobutyrate |
| cyclal C | 2,4-dimethylcyclohex-3-ene-1-carbaldehyde |
| cyclaprop | hexahydro-4,7-methano-indenyl propionate |
| cyclohexylethyl | 2-cyclohexylethyl acetate |
| acetate | |
| cyclopentol | 2-pentylcyclopentan-1-ol |
| cymal | (2R,3R,4S,5S,6R)-2-[(2R,3S,4R,5R,6R)-6-(6- |
| | cyclohexylhexoxy)-4,5-dihydroxy-2-(hydroxymethyl)oxan- |
| | 3-yl]oxy-6-(hydroxymethyl)oxane-3,4,5-triol |
| damascenone | (E)-1-(2,6,6-trimethyl-1-cyclohexa-1,3-dienyl)but-2-en-1-one |
| datilat | 1-cyclohexylethyl (E)-but-2-enoate |
| decyl aldehyde | decanal |
| delta damascone | (E)-1-(2,6,6-trimethyl-1-cyclohex-3-enyl)but-2-en-1-one |
| delta muscenone | (5E)-3-methylcyclopentadec-5-en-1-one |
| dihydro-alpha- | 4-(2,6,6-trimethyl-1-cyclohex-2-enyl)butan-2-one |
| ionone | |
| dihydroeugenol | 2-methoxy-4-propylphenol |
| dihydroisojasmonate | methyl 2-hexyl-3-oxocyclopentane-1-carboxylate |
| dihydromyrcenol | 2,6-dimethyloct-7-en-2-ol |
| dimethyl octenone | 4,7-dimethyloct-6-en-3-one |
| dupical | 4-(octahydro-4,7-methano-5H-inden-5-yliden)butanal |
| elintaal | acetaldehyde ethyl linalyl acetal |
| ethyl 3,7-dimethyl-2,6- | ethyl 3,7-dimethyl-2,6-octadienoate |
| octadienoate | |
| ethyl beta- | ethyl 2,6,6-trimethylcyclohexa-1,3-diene-1-carboxylate |
| safranate | |
| ethyl hexanoate | 2-ethylhexanoate |
| ethyl linalool | (6E)-3,7-dimethylnona-1,6-dien-3-ol |
| ethyl methyl | ethyl 2-methylbutanoate |
| butyrate | |
| ethyl methyl | ethyl 2-methylpentanoate |
| pentanoate | |
| ethyl myristate | ethyl tetradecanoate |
| ethyl nonanoate | ethyl nonanoate |
| ethyl-3- | ethyl 3-phenyloxirane-2-carboxylate |
| phenylglycidate | |
| ethylene brassylate | 1,4-dioxacycloheptadecane-5,17-dione |
| eucalyptol | 1,3,3-trimethyl-2-oxabicyclo[2,2,2]octane |
| eucalyptus | [essential oil] |
| exaltolide | oxacyclo-hexadecan-2-one |
| floralozone | 3-(4-ethylphenyl)-2,2-dimethylpropanal |
| freskomenthe | 2-butan-2-ylcyclohexan-1-one |
| fructalate | 1,4-cyclohexandicarboxylic acid, diethyl ester |
| fruitate | (3aalpha,4beta,7beta,7aalpha)-octahydro-4,7-methano- |
| | 3aH-indene-3a-carboxylic acid ethyl ester |
| frutene | hexahydro-4-7, menthano-1H-inden-6-yl propionate |
| gamma damascone | 2-butenon-1-one,1-(2,6-dimethyl-6-methylencyclohexyl)- |
| gamma ionone | (E)-4-(2,2-dimethyl-6-methylidenecyclohexyl)but-3-en-2-one |
| gamma terpinene | 1-methyl-4-propan-2-ylcyclohexa-1,4-diene |
| gamma | 5-heptyloxolan-2-one |
| undecalactone | |
| geraniol | 3,7-dimethylocta-2,6-dien-1-ol |
| geranyl acetate | [(2E)-3,7-dimethylocta-2,6-dienyl] acetate |
| geranyl caprylate | [(2E)-3,7-dimethylocta-2,6-dienyl] octanoate |
| givescone | ethyl 2-ethyl-6,6-dimethylcyclohex-2-ene-1-carboxylate |
| g-terpineol acetate | (4-methyl-1-propan-2-yl-1-cyclohex-2-enyl) acetate |
| gyrane | 2-butyl-4,6-dimethyl-5,6-dihydro-2H-pyran |
| habanolide | oxacyclohexadecen-2-one |
| helvetolide | 1-propanol,2-[1-(3,3-dimethyl-cyclohexyl)ethoxy]-2- |
| | methyl-propanoate |
| heptyl acetate | 1-heptyl acetate |
| hexyl acetate | 1-hexyl acetate |
| hexyl isobutyrate | hexyl 2-methylpropanoate |
| hyacinth body | (2-(1-ethoxyethoxy)ethyl)benzene |
| indoflor | 4,4a,5,9b-tetrahydroindeno[1,2-d][1,3]dioxine |
| intreleven aldehyde | undec-10-enal |
| ionone gamma | 3-methyl-4-(2,6,6-trimethyl-2-cyclohexen-1-yl)-3-buten-2-one |
| methyl | |
| iso E super | 1-(1,2,3,4,5,6,7,8-octahydro-2,3,8,8-tetramethyl-2- |
| | naphthalenyl)-ethan-1-one |
| iso gamma super | 7-acetyl,1,2,3,4,5,6,7-octahydro-1,1,6,7,-tetra methyl |
| | naphtalene |
| isoamyl salicylate | 3-methylbutyl 2-hydroxybenzoate |
| isobornyl acetate | [(1R,4S,6R)-1,7,7-trimethyl-6-bicyclo[2.2.1]heptanyl] acetate |
| isobornyl isobutyrate | [(1R,4R,6R)-1,7,7-trimethyl-6-bicyclo[2.2.1]heptanyl] |
| (abierate cn) | 2-methylpropanoate |
| isobornyl propionate | (1,7,7-trimethyl-5-bicyclo[2.2.1]heptanyl) propanoate |
| isobutyl caproate | 2-methylpropyl hexanoate |
| isoeugenyl acetate | [2-methoxy-4-[(E)-prop-1-enyl]phenyl] acetate |
| isojasmone | 2-hexylcyclopent-2-en-1-one |
| isomenthone | 5-methyl-2-propan-2-ylcyclohexan-1-one |
| isononyl acetate | 7-methyloctyl acetate |
| isopropyl-2- | propan-2-yl 2-methylbutanoate |
| methylbutyrate | |
| koavone | 3,4,5,6,6-pentamethylheptenone-2 |
| koumalactone | hexahydro-3,6-dimethyl-2(3H)-benzofuranone |
| labienoxime | 2,4,4,7-tetramethyl-6,8-nonadiene-3-one oxime |
| lauryl acetate | dodecyl acetate |
| lavandin | [essential oil] |
| lemonile | 3,7-dimethylnona-2,6-dienenitrile |
| liffarome | [(Z)-hex-3-enyl] methyl carbonate |
| lilial | 2-methyl-3-(4-tert-butylphenyl)propanal |
| linalool | 3,7-dimethylocta-1,6-dien-3-ol |
| linalyl acetate | 3,7-dimethylocta-1,6-dien-3-yl acetate |
| linalyl butyrate | 3,7-dimethylocta-1,6-dien-3-yl butanoate |
| linalyl formate | 3,7-dimethylocta-1,6-dien-3-yl formate |
| linalyl isobutyrate | 3,7-dimethylocta-1,6-dien-3-yl 2-methylpropanoate |
| linalyl propionate | 3,7-dimethylocta-1,6-dien-3-yl propanoate |
| maceal | 3-methyl-7-propan-2-ylbicyclo[2.2.2]oct-2-ene-5- |
| | carbaldehyde |
| majantol | 2,2-dimethyl-3-(3-methylphenyl)propan-1-ol |
| mefloral | 3-(4-tert-butylphenyl)butanal |
| melonal | 2,6-dimethylhept-5-enal |
| menthol | 5-methyl-2-propan-2-yl-cyclohexan-1-ol |
| methyl beta | 1-(2,6,6-trimethyl-1-cyclohexenyl)pent-1-en-3-one |
| ionone | |
| methyl | methyl 3-oxo-2-pentylcyclopentaneacetate |
| dihydrojasmonate | |
| methyl myristate | methyl tetradecanoate |
| methyl nonyl | 2-methylundecanal |
| acetaldehyde | |
| methyl octyl | 2-methyldecanal |
| acetaldehyde | |
| methyl | 1,1-dimethoxy-2,2,5-trimethyl-4-hexene |
| pamplemousse | |
| myraldyl acetate | [(1S)-3-(4-methylpent-3-enyl)-1-cyclohex-3-enyl]methyl |
| | acetate |
| nectaryl | 2-(2-(4-methyl-3-cyclohexen-1-yl)propyl)cyclo-pentanone |
| neobutenone | 4-penten-1-one, 1-(5,5-dimethyl-1-cyclohexen-1-yl |
| neohivernal | 1H-indene-ar-propanal,2,3,-dihydro-1,1-dimethyl-(9CI) |
| nerolin | 2-ethoxynaphthalene |
| nonyl aldehyde | nonanal |
| nopyl acetate | 2-(7,7-dimethyl-4-bicyclo[3.1.1]hept-3-enyl)ethyl acetate |
| octyl aldehyde | octanal |
| orange flower | 4-(1-methoxy-1-methylethyl)-1-methylcyclohexene |
| ether | |
| o-tert-butylcyclohexyl | (2-tert-butylcyclohexyl) acetate |
| acetate | |
| ozofleur | (E)-1-ethoxy-4-(2-methylbutan-2-yl)cyclohexane |
| parmavert | 1,1-dimethoxynon-2-yne |
| patchouli | [essential oil] |
| peonile | 2-cyclohexylidene-2-phenylacetonitrile |
| pharaone | 2-cyclohexyl-1,6-heptadien-3-one |
| phenyl ethyl dimethyl | 4-cyclohexyl-2-methylbutan-2-ol |
| carbinol | |
| phenyl ethyl phenyl | 2-phenylethyl 2-phenylacetate |
| acetate | |
| pomarose | (2E, 5E/Z)-5,6,7-trimethyl octa-2,5-dien-4-one |
| precyclemone B | 1-methyl-3-(4-methylpent-3-enyl)cyclohex-3-ene-1-carbaldehyde |
| romascone | methyl 2,2-dimethyl-6-methylidenecyclohexane-1-carboxylate |
| rosamusk | 1-(3,3-dimethylcyclohexyl)ethyl acetate |
| rose oxide | 4-methyl-2-(2-methylprop-1-enyl)oxane |
| spirogalbanone | 1-spiro(4.5)-7-decen-7-yl-4-penten-1-one |
| tabanone | 4-(2-butenylidene)-3,5,5-trimethylcyclohex-2-en-1-one |
| terpineol | 2-(4-methyl-1-cyclohex-3-enyl)propan-2-ol |
| terpinolene | 4-isopropylidene-1-methyl-cyclohexene |
| terpinyl acetate | 2-(4-methyl-1-cyclohex-3-enyl)propan-2-yl acetate |
| tetrahydromuguol | 3,7-dimethyloctan-3-ol |
| tetrahydrolinalool | 3,7-dimethyloctan-3-ol |
| tetrahydrolinalyl | 3,7-dimethyloctan-3-yl acetate |
| acetate | |
| trifernal | 3-phenylbutanal |
| triplal | (2,5-dimethyl-4-oxofuran-3-yl) acetate |
| undecavertol | 4-methyl-3-decen-5-ol |
| undecylenic aldehyde | undec-10-enal |
| vanillin isobutyrate | (4-formyl-2-methoxyphenyl) 2-methylpropanoate |
| veloutone | 2,2,5-trimethyl-5-pentylcyclopentan-1-one |
| verdol | 2-tert-butylcyclohexan-1-ol |
| verdox | (2-tert-butylcyclohexyl) acetate |
| vertenex | 4-tert-butylcyclohexyl acetate |
| vertofix | 1-(3-methyl-7-propan-2-yl-6-bicyclo[2.2.2]oct-3-enyl)ethanone |
| vetivert acetate | (4,8-dimethyl-2-propan-2-ylidene-3,3a,4,5,6,8a-hexahydro-1H- |
| | azulen-6-yl) acetate |
| violiff | [(4Z)-1-cyclooct-4-enyl] methyl carbonate |
| yara yara | methyl beta naphtyl ether |

### Polymer

The polymer of the present invention may be any suitable polymer known in the art. A non-limiting example of a suitable polymer comprises a copolymer of ethylene with at least another monomer comprising at least a heteroatom. The phrase “monomer comprising at least a heteroatom” includes all those monomers which comprise at least a C—X linkage in the molecule wherein X is not C or H. In one example, the C—X linkage is a polar linkage. In another example, the carbon atom is linked to an N, S, F, Cl or O atom. In yet another example, the polar linkage is part of a carbonyl group, for example part of an ester group.

In one example, the polymer may be any suitable polymer that exhibits a lower melting point after dissolving of a highly compressible fluid described herein.

In still another example, the polymer may be any suitable polymer that dissolves a highly compressible fluid described herein.

Non-limiting examples of monomers that comprise at least a heteroatom include vinyl acetate, vinyl alcohol, methyl acrylate, ethyl acrylate, butyl acrylate, acrylic acid and salts formed therefrom, methacrylic acid and salts formed therefrom, maleic anhydride, glycidyl methacrylate, carbon monoxide, and mixtures thereof. The monomer comprising at least a heteroatom in the copolymers suitable for the present invention may be present in the copolymer of ethylene at a level of from about 10% to about 90% and/or at least 14% and/or at least 18% by weight of the copolymer.

Suitable copolymers for the present invention can be both block and non-block copolymers, grafted copolymers, copolymers with side chains, or crosslinks and copolymers where ethylene monomers are randomly copolymerized with monomers comprising at least a heteroatom.

Non-limiting examples of suitable water-insoluble copolymers of ethylene according to the present invention include ethylene-vinyl ester copolymers, ethylene-acrylic ester copolymers, ethylene-methacrylic ester copolymers, ethylene-acrylic acid copolymers and their salts, ethylene-methacrylic acid copolymers and their salts, ethylene-vinyl ester-acrylic acid copolymers, ethylene-vinyl ester-methacrylic acid copolymers, ethylene-vinyl ester-maleic anhydride copolymers, ethylene-acrylic ester-maleic anhydride copolymers, ethylene-vinyl ester-glycidyl methacrylate copolymers, ethylene-acrylic ester-glycidyl methacrylate copolymers, ethylene-maleic anhydride copolymers, ethylene-glycidyl methacrylate copolymers.

In one example, a suitable water-insoluble copolymer of ethylene comprises ethylene-vinyl acetate copolymer, such as those sold under the trade names Elvax® by Dupont, Evathane® by Atofina, Escorene® by Exxon and Levapren® and Levamelt® by Bayer and ethylene-acrylic ester copolymers such as those sold under the trade name Lotryl® by Atofina.

Other suitable water-insoluble polymers for the present invention include thermoplastic polymers, such as polyesters and/or nylons.

In another example, the polymer may comprise a water-soluble polymer, which may comprise a copolymer and/or derivative thereof, selected from the group consisting of: polyvinyl alcohols, modified polyvinyl alcohols, polyvinyl pyrrolidone, polyvinyl alcohol copolymers, such as polyvinyl alcohol/polyvinyl pyrrolidone and polyvinyl alcohol/polyvinyl amine, partially hydrolyzed polyvinyl acetate, polyalkylene oxides, such as polyethylene oxide, polyethylene glycols, acrylamide, acrylic acid, alkyl celluloses, such as methyl cellulose, ethyl cellulose and propyl cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and modified starch, gelatin, alginates, xyloglucans, other hemicellulose polysaccharides including xylan, glucuronoxylan, arabinoxylan, mannan, glucomannan and galactoglucomannan, and natural gums such as pectin, xanthan, and carrageenan, locus bean, arabic, tragacanth, and mixtures thereof. In another example, the polymer, which comprises a copolymer and/or derivative thereof, is selected from the group consisting of: polyacrylates, for example sulfonated polyacrylates, acrylate copolymers, alkylhydroxy celluloses, such as methylcellulose, carboxymethylcellulose sodium-modified carboxymethylcellulose, dextrin, ethylcellulose, propylcellulose, hydroxyethyl cellulose, hydroxypropyl methylcellulose, maltodextrin, polymethacrylates and mixtures thereof. In still another example, polymer is selected from the group consisting of: polyvinyl alcohols, polyvinyl copolymers, hydroxypropyl methyl cellulose (HPMC); and mixtures thereof.

In another example, due to the relatively low processing temperatures of the PGSS processes, polymers that are negatively impacted by the relatively high processing temperatures present in known volatile material particle formation processes, such as coacervation, interfacial polymerization and poly-condensation processes, can be used to make the particles of the present invention. In addition, the volatile materials are often thermally sensitive and lower temperatures encountered with PGSS process avoid their flashing and degradation.

### Lipophilic Material

The lipophilic material comprises a lipophilic agent. Non-limiting examples of suitable lipophilic agents include ester lipids, hydrocarbon lipids, silicone lipids, fatty alcohols, fatty acids, and mixtures thereof.

Non-limiting examples of suitable ester lipids include lipids that have at least one ester group in the molecule. One type of common ester lipids useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate, sucrose esters (such as sucrose esters derived from fatty acids) and polyesters, sorbitol ester, and the like.

In one example, the lipophilic material comprises glyceryl monooleate.

In another example, the lipophilic material comprises paraffin and/or a microcrystalline wax.

Another type of ester lipid suitable for the present invention includes triglycerides and modified triglycerides, and mixtures thereof. These include vegetable oils such as jojoba, soybean, canola, sunflower, safflower, rice bran, avocado, almond, olive, sesame, persic, castor, coconut, and mink oils. Synthetic triglycerides can also be employed. Modified triglycerides include materials such as ethoxylated and maleated triglyceride derivatives. Proprietary ester blends such as those sold by Finetex as Finsolv are also suitable, as is ethylhexanoic acid glyceride.

A third type of ester lipid is liquid polyester formed from the reaction of a dicarboxylic acid and a diol. Examples of polyesters suitable for the present invention are the polyesters marketed by ExxonMobil under the trade name PURESYN ESTER®.

Non-limiting examples of suitable hydrocarbon lipids, which may be liquid or semi-solid hydrocarbons, include linear and branched oils such as liquid paraffin, squalene, squalene, mineral oil, low viscosity synthetic hydrocarbons such as polyalphaolefin sold by ExxonMobil under the trade name of PURESYN PAO and polybutene under the trade name PANALANE or INDOPOL, and mixtures thereof. Light (low viscosity), highly branched hydrocarbon oils are also suitable.

Petrolatum is an example of a hydrocarbon lipid that is suitable for the present invention. Its semi-solid nature can be controlled both in production and by the formulator through blending with other oils or fractionating to remove one or more of the hydrocarbon components from the blend, such as eliminating lower chains (for example C₂₀-C₃₆). Petrolatum is often described as a “complexed mixture of cyclic, branched, and linear hydrogenated hydrocarbon oils and waxes commonly referred to as mineral oils, paraffin and microcrystalline waxes”. In one example, the petrolatum is void or significantly void of all lower chains (for example C₂₀-C₃₆) white oils & cyclic paraffins, which have been replaced with a higher viscosity mineral oil having longer chains (for example C₄₀-C₅₀), for example Hydrobrite 1000, which is commercially available from R. E. Carroll, Inc., Trenton, N.J. Additionally, the level of microcrystalline wax (having chain lengths of from about C₃₀-C₇₅) can be increased to stabilize the oils at room temperature (about 23° C.) and to provide the needed lipid structure at elevated temperatures. This petrolatum may exhibit a melting point of from about 135° F. to about 155° F. and a viscosity at 210° F. of 80 centipoise or greater as measured by a Brookfield Viscometer.

Another example of a suitable petrolatum is known in the art as Super White Petrolatum. It exhibits a melting point of from about 130° F. to about 140° F. and a viscosity at 210° F. of less than 80 centipoise as measured by a Brookfield Viscometer.

In another example, a polymer-modified petrolatum, such as Versagel P200 commercially available from Penreco, Houston, Tex., is suitable for use in the present invention. This petrolatum contains a polymer thickening agent, which may serve to increase the viscosity of the petrolatum.

Non-limiting examples of suitable silicone lipids include linear and cyclic polydimethyl siloxane, organo functional silicones (alkyl and alkyl aryl), and amino silicones, and mixtures thereof.

Non-limiting example of suitable fatty alcohols include liquid fatty alcohols having from about 10 to about 30 carbon atoms. These liquid fatty alcohols may be straight or branched chain alcohols and may be saturated or unsaturated alcohols. Liquid fatty alcohols are those fatty alcohols which are liquid at about 25° C. Non-limiting examples of these compounds include oleyl alcohol, palmitoleic alcohol, isostearyl alcohol, isocetyl alcohol, and mixtures thereof.

Non-limiting examples of suitable fatty acids include liquid fatty acids having from about 10 to about 30 carbon atoms. These fatty acids can be straight or branched chain acids and can be saturated or unsaturated. Suitable fatty acids include, for example, oleic acid, linoleic acid, isostearic acid, linolenic acid, ethyl linolenic acid, arachidonic acid, ricinolic acid, and mixtures thereof.

### Tackifier

A tackifier otherwise called “a tackifier resin” or “tackifying resin” are materials which are commonly sold as such and are used in hot melt adhesives in order to improve the adhesive properties of the material present in the polymer and/or solution. A good tackifier is compatible with the copolymer, has a low molecular weight with respect to the copolymer and has a Tg (glass transition temperature) which is higher than that of the copolymer, so that when they are introduced into the particle or solution, the Tg of particle or solution is increased. Non-limiting examples of suitable tackifiers for the present invention include thermoplastic materials, stable to at least 200° C., amorphous or glassy at 23° C.±2.2° C., and having a Tg higher than 50° C. and/or between 80° C. and 125° C. In one example, one or more tackifiers exhibit a molecular weight of between 500 and 2000 Daltons.

In one example, the tackifiers comprise organic chemicals with polycyclic structures, which are not aliphatic hydrocarbons. In another example, the tackifiers are aromatic tackifiers and/or tackifiers that comprise oxygen atoms. In still another example, the tackifiers are rosin and its derivatives which are solid at 23° C.±2.2° C.

### Plasticizer

A compatible plasticizer or blend of plasticizers can be optionally present in the particles and/or solution according to the present invention up to a concentration of about 10% by weight of the total weight of the particle and/or solution. The term “compatible” indicates a material which can be stably formulated in the matrix without forming a separated phase. The term “plasticizer,” as known to those skilled in the art of thermoplastic polymeric materials, defines a class of materials which are introduced into polymeric materials to make them softer and more flexible. More specifically plasticizers cause an increase in the flexibility and workability, brought about by a decrease in the glass-transition temperature, Tg, of the polymer.

### Phase Change Solvent

A phase change solvent may be included in the particles and/or solution according to the present invention up to a concentration of about 10% by weight of the total weight of the particle and/or solution. Non-limiting examples of suitable phase change solvents include phase change solvents having a phase change in a temperature range from 40° C. to 250° C.

R′—P_{y}-(Q-Pₓ)ₙ₋₁-Q-P_{y}—R (I)

R′—P_{y}-(Q-Pₓ)ₙ—R (II)

R′-(Q-Pₓ)ₙ—R (III)

R′-(Q-Pₓ)ₙ₋₁-Q-P_{y}—R (IV-a)

R′-(Q-Pₓ)ₙ₋₁-Q-R (IV-b)

R′—P_{y}—(W—R″)ₙ₋₁—W—P_{y}—R (V)

R′—P_{y}—(W—R″)ₙ—R (VI)

R′—(W—R″)ₙ₋₁—W—P_{y}—R (VII)

R′—P_{y}—(W—R″—W′—R′″)ₙ₋₁—W—P_{y}—R (VIII)

R′—P_{y}—(W—R″-W′—R′″)ₙ—R (IX)

R′—(W—R″—W′—R′″)ₙ₋₁—W—P_{y}—R (X)

For formulas (I)-(IV-b), Q is a substituted or unsubstituted difunctional aromatic moiety. Exemplary Q groups are terephthalic, naphthalic, phenolic, phenyl, or biphenyl or mixtures thereof. P is CH₂; R and R′ may be the same or different and are independently selected from the group consisting of H, CH₃, COOH, CONHR₁, CONR₁R₂, NHR₃, NR₃R₄, hydroxy, and C₁-C₃₀ alkoxy; wherein R₁, R₂, R₃ and R₄ are independently H or linear or branched alkyl from C₁-C₃₀; x is an integer from 1 to 30; y is an integer from 1 to 30; and n is an integer from 1 to 7. Q may be substituted on the aromatic ring with one or more substituents selected from the group consisting of H, C₁-C₃₀ alkyl, COOH, CONHR₅, CONR₅R₆, NHR₇, NR₇R₈, hydroxy, C₁-C₃₀ alkoxy, SO₃H, and halogen; wherein R₅, R₆, R₇ and R₈ are independently H or linear or branched alkyl from C₁-C₃₀.

### Highly Compressible Fluid

The term “highly compressible fluid” as used herein is defined by way of the reduced temperature (T_{reduced}) and the reduced pressure (p_{reduced}) of the fluid (in pure form) used as a highly compressible fluid. With

$${T}_{reduced} = \frac{T [ K ]}{{T}_{critical} [ K ]} and$$

$${P}_{reduced} = \frac{p [ bar ]}{{p}_{critical} [ bar ]}$$

a fluid is defined in the present application as being highly compressible if its reduced temperature is in a range of 0.5 to 2.0 and/or in the range of 0.8 to 1.7 and its reduced pressure is between 0.3 and 8.0. The highly compressible fluid may thus be subcritical with regard to temperature and supercritical with regard to pressure or vice versa or may be subcritical with regard to both temperature and pressure or may be supercritical with regard to both temperature and pressure.

Suitable highly compressible fluids are a whole series of substances. Non-limiting examples of suitable highly compressible fluids include carbon dioxide, short-chain alkanes, dinitrogen monoxide, nitrogen and mixtures thereof. However, in principle, it is possible to use the vapor phase of any of the substances mentioned in Table 2, and mixtures of these substances, as highly compressible fluid.

TABLE 2

| | Boiling | Critical | Critical | Critical |
|---|---|---|---|---|
| | Point | Temperature | Pressure | Density |
| Compound | (° C.) | (° C.) | (bar) | (kg/m³) |
| CO₂ | −78.5 | 31.3 | 72.9 | 0.448 |
| NH₃ | −33.35 | 132.4 | 112.5 | 0.235 |
| H₂O | 100.00 | 374.15 | 218.3 | 0.315 |
| N₂O | −88.56 | 36.5 | 71.7 | 0.45 |
| CH₄ | −164.00 | −82.1 | 45.8 | 0.2 |
| Ethane | −88.63 | 32.28 | 48.1 | 0.203 |
| Ethylene | −103.7 | 9.21 | 49.7 | 0.218 |
| Propane | −42.1 | 96.67 | 41.9 | 0.217 |
| Propylene | −47.4 | 91.9 | 45.4 | — |
| n-Butane | −0.5 | 152.0 | 37.5 | — |
| i-Butane | −11.7 | 134.7 | 35.9 | — |
| n-Pentane | 36.1 | 196.6 | 33.3 | 0.232 |
| Benzene | 80.1 | 288.9 | 48.3 | 0.302 |
| Methanol | 64.7 | 240.5 | 78.9 | 0.272 |
| Ethanol | 78.5 | 243.0 | 63.0 | 0.276 |
| Isopropanol | 82.5 | 235.3 | 47.0 | 0.273 |
| Isobutanol | 108.0 | 275.0 | 42.4 | 0.272 |
| Chlorotrifluoro- | −31.2 | 28.0 | 38.7 | 0.579 |
| methane | | | | |
| Monofluoromethane | 78.4 | 44.6 | 58.0 | 0.3 |
| Toluene | 110.6 | 320.0 | 40.6 | 0.292 |
| Pyridine | 115.5 | 347.0 | 55.6 | 0.312 |
| Cyclohexane | 80.74 | 280.0 | 40.2 | 0.273 |
| Cyclohexanol | 155.65 | 391.0 | 25.8 | 0.254 |
| o-Xylene | 144.4 | 357.0 | 35.0 | 0.284 |
| CO₂ | −78.5 | 31.3 | 72.9 | 0.448 |
| NH₃ | −33.35 | 132.4 | 112.5 | 0.235 |
| H₂O | 100.00 | 374.15 | 218.3 | 0.315 |
| N₂O | −88.56 | 36.5 | 71.7 | 0.45 |
| CH₄ | −164.00 | −82.1 | 45.8 | 0.2 |
| Ethane | −88.63 | 32.28 | 48.1 | 0.203 |
| Ethylene | −103.7 | 9.21 | 49.7 | 0.218 |
| Propane | −42.1 | 96.67 | 41.9 | 0.217 |
| Propylene | −47.4 | 91.9 | 45.4 | — |
| n-Butane | −0.5 | 152.0 | 37.5 | — |
| i-Butane | −11.7 | 134.7 | 35.9 | — |
| n-Pentane | 36.1 | 196.6 | 33.3 | 0.232 |
| Benzene | 80.1 | 288.9 | 48.3 | 0.302 |
| Methanol | 64.7 | 240.5 | 78.9 | 0.272 |
| Ethanol | 78.5 | 243.0 | 63.0 | 0.276 |
| Isopropanol | 82.5 | 235.3 | 47.0 | 0.273 |
| Isobutanol | 108.0 | 275.0 | 42.4 | 0.272 |
| Chlorotrifluoro- | −31.2 | 28.0 | 38.7 | 0.579 |
| methane | | | | |
| Monofluoromethane | 78.4 | 44.6 | 58.0 | 0.3 |
| Toluene | 110.6 | 320.0 | 40.6 | 0.292 |
| Pyridine | 115.5 | 347.0 | 55.6 | 0.312 |
| Cyclohexane | 80.74 | 280.0 | 40.2 | 0.273 |
| Cyclohexanol | 155.65 | 391.0 | 25.8 | 0.254 |
| o-Xylene | 144.4 | 357.0 | 35.0 | 0.284 |

One or more of the materials within the solution into which the highly compressible fluid is dissolved may initially be a solid rather than a liquid. If it is a solid, then the solid is transformed into a liquid as a result of the highly compressible fluid dissolving within the solution under pressure of at least 50 bars. The mass ratio between the highly compressible fluid and the solution into which the highly compressible fluid is dissolved may be from about 0.1:1 to about 4:1.

In order to fully understand the present invention it is necessary to appreciate what is meant by dissolving or solubilizing a highly compressible fluid in a liquid or a solid substance.

### Process for Making Particles

The particles of the present invention may be produced by a PGSS process. In one example of the present invention, a first material, for example a polymer may be mixed with a second material, for example a volatile material, such as a perfume, to form a solution or an emulsion. From here forward solution and/or emulsion are used interchangeably. The first and second materials are under conditions such that they are present in the solution in their liquid states. Optionally, the solution can be pressurized to a pressure of at least 50 bars thus producing a pressurized solution. A highly compressible fluid may then be dissolved or partially dissolved in the solution thereby also pressurizing the system to 50 bar or higher. The pressurized solution is then rapidly depressurized as the solution is sprayed through a spray nozzle. During the depressurization and/or spraying, the highly compressible fluid is released from the solution and particles comprising the first material and second material are produced.

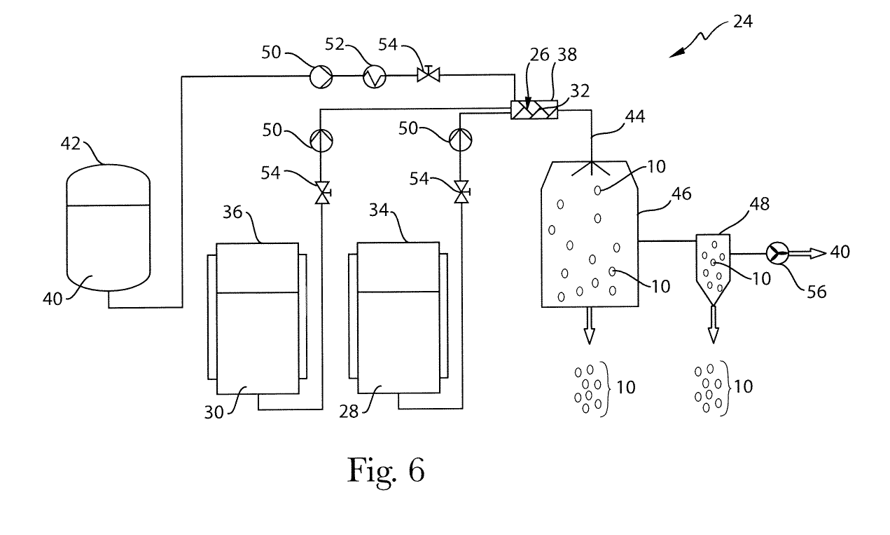

As shown in FIG. 6 a PGSS process 24 according to the present invention produces a particle and/or a plurality of particles 10. The PGSS process 24 comprises providing a pressurized solution 26 comprising at least a first material 28, such as a volatile material, and a second material 30, such as a polymer. The first material 28 and second material 30 are mixed together to form a solution 32. The first material 28 may be sourced from a first storage vessel 34 and the second material 30 may be sourced from a second storage vessel 36. The solution 32 is pressurized and under conditions such that the first and second materials 28, 30 are in their liquid states. The first and second materials 28, 30 may be mixed together in a mixer 38, such as a static mixer, to form the solution 32.

A highly compressible fluid 40, which may be a liquid or a gas, is dissolved in the solution 32. The highly compressible fluid 40 may be sourced from a third storage vessel 42 and mixed with the first and second materials 28, 30 in the mixer 38.

After at least a portion of the highly compressible fluid 40 is dissolved into the solution 32, the solution 32 is then depressurized by spraying through one or more spray nozzles 44, such as within a spray tower 46. During the spraying operation, the solution 32 is depressurized and particles 10 are produced. The highly compressible fluid 40 is released from the solution 32 during the spraying operation. The highly compressible fluid 40 may have particles 10 entrained therein so it may be necessary to collect the particles 10 that are entrained in the highly compressible fluid 40. This collection may occur by passing the highly compressible fluid 40 through a cyclone filter 48 in order to separate the particles 10 from the highly compressible fluid 40 and increase the yield of the particles 10 produced by the PGSS process 24.

As shown in FIG. 6, the various components used in the PGSS process are fluidly connected to one another by any suitable piping, conduits, tubes, and the like. In one example, suitable pumps 50 may be used to help the flow of the materials within the process. In addition, a heat exchanger 52 may be utilized for one or more of the materials, for example the highly compressible gas 40. In still another example, stopcocks 54 may be used to manage the flow of the materials within the process. In still another example, a blower or fan 56 may be utilized within the process in order to help remove the highly compressible fluid 40 from the neat particles 10 produced in the process.

In addition to collecting the neat particles 10 as they are produced, the particles 10 may be collected in a slurry or suspension. In another example, the particles 10 may be mixed with a carrier in a Concentrated Powder Form (CPF) technology process. For example, a carrier, such as a waxy, powdery carrier is admixed into a stream of the particles 10 such that the particles contact and associate with the carrier to form a particle-charged carrier. The particle-charged carrier can then be collected. In one example, the average particle size of the carrier is less than 1 mm and/or less than 500 μm and/or less than 300 μm and/or less than 100 μm and/or less than 50 μm as measured by the Particle Size Test Method described herein. In other examples, the carrier may be a waxy or non-waxy solid at 23° C.±2.2° C. or a mineral, including silica or calcium carbonate.

In another example, the particles 10 and/or particle-charged carriers may be coated with a coating material to control the release of materials from the particles 10 and/or particle-charged carriers and/or influence the stability, such as shelf life, of the particles 10 and/or particle—charged carriers. The coating process may occur in a fluidized bed coater and/or a spray coating application process. In one example, coatings may be lipophilic or waxy materials such as paraffin. In another example, coatings may be aliphatic polymers such as polyethylene or polyethylene wax. Other non-limiting examples include poly(methyl methacrylate), or PMMA; poly(vinyl alcohol), or PVOH; poly(ethylene glycol), or PEG; and poly(ethylene oxide), or PEO. Non-limiting examples of suitable coating processes and/or materials are described in U.S. Pat. Nos. 6,221,826 and 7,338,928, both of which are incorporated herein by reference.

The PGSS process of the present invention thus produces particles from a solution, such as a liquid solution, producing a higher loading of volatile material in the polymer matrix in the particle structure, and a far lower highly compressible fluid content than was previously considered necessary for other known particle production processes using compressible fluids, such as RESS (rapid expansion from supercritical solutions). The cooling of the solution is so great, despite the unusually low highly compressible fluid content and high solution (incompatible materials) content, that the temperature falls below the solidification point of the solution to be treated downstream of the spray nozzle (decompression device). On decompression of a highly compressible fluid-containing solution in a suitable device, e.g. a commercially obtainable high-pressure spray nozzle, the highly compressible fluid is returned to the gaseous state and the solution (incompatible materials) to be treated precipitates as particles.

For the solidification point to be reached upon decompressing the solution it is necessary to comply with certain conditions. The melting point of the highly compressible fluid used should be at least 40 K and/or at least 80 K, and/or at least 100 K lower than the melting point of at least one and/or at least two and/or all the materials within the solution.

To assure that the cooling effect upon decompressing the solution is pronounced enough for particles to form there has to be a certain minimum amount of highly compressible fluid dissolved in the solution. Depending on the solution to be treated and the type of highly compressible fluid used that minimum amount of highly compressible fluid dissolved in the solution may be from about 5% to about 90% and/or from about 8% to about 70% and/or from about 10% to about 50% by weight of the solution. Further, the temperature of the highly compressible fluid-containing solution before decompression should be in the region of up to 50 K and/or up to 20 K and/or up to 10 K above or below the melting point of at least one and/or at least two and/or all of the materials within the solution under atmospheric pressure.

### Test Methods

Unless otherwise indicated, all tests described herein, including those described under the Definitions section and the following test methods, are conducted on samples that have been conditioned in a conditioned room at a temperature of 23° C.±2.2° C. and a relative humidity of 50%±10% for 2 hours prior to the test. Further, all tests are conducted in such a conditioned room.

### Particle Size Test Method

The average particle size of a particle is measured using a Horiba LA-910 commercially available from Horiba International Corporation of Irvine, Calif.

One skilled in the art knows that the suitable and appropriate operating conditions for the Horiba LA-910 can be found by running one or more pilot runs on the Horiba LA-910 for the particle sample. Visually, one skilled in the art can determine whether the particle sample is bimodal or unimodal regarding particle size. If the particle sample contains agglomerates, then one of skill in the art will utilize ultrasonics to break up the agglomerates before measuring the particle size. During the pilot run(s), whether the particle sample is bimodal or unimodal can be determined. During the pilot runs, one skilled in the art can determine the appropriate agitation and circulation speed, and if the average particle size from the particle sample is less than 10 μm, can obtain the relative refractive index from Horiba's database.

Follow the Horiba LA-910 Instrument manual for setup and software use instructions. Obtain the relative refractive index for the particle sample to be tested from the Horiba refractive index database.

Input the appropriate measurement conditions into the instrument: Agitation and Circulation Speed—obtained from pilot run(s); Sampling Times 25; Standard Distribution; Dispersant Tank B; Dispersant Volume 200 ml; Dispersant Volume per Step 10 ml; Dilution Point 10%; Rinse Circulation Time 10 seconds; Rinse Repeat Times 1; Rinsing Volume 100 ml; Relative Refractive Index; Good Range Lower Limit 88%; and Good Range Upper Limit 92%.

Drain the cell of the instrument and add 150 mL of the dispersant to the cell and circulate, sonicate for 2 minutes, and agitate. If the cell looks clean and the background reading looks flat, run a blank by pressing “Blank.” Add the solid additive sample to be tested to the cell while the dispersant is agitating and circulating. Continue to add the solid additive sample slowly until the % Transmission of the laser is 90+/−2 (around 1 mL). Allow the particle sample to circulate through the cell for 2 minutes. After the particle sample has circulated for 2 minutes, press “Measure” to analyze the particle sample. Once the particle sample is analyzed, print the graph and table. Press “Drain” to drain the cell. Rinse the system three times with deionized water using agitation and sonication for 30 seconds each time. For subsequent particle samples, repeat steps 2-10. The laser alignment (four triangles) should be checked between particle samples. The results are reported as follows: 1) a standard resolution histogram for a unimodal distribution or a sharp resolution histogram for a multi-modal distribution; and 2) the Average Particle Size (Mean Diameter).

### Contact Angle Test Method

The contact angle of a material is measured using a DAT 1100 FIBRO system commercially available from Thwing-Albert Instrument Company of West Berlin, N.J.

The syringe and tubing of the DAT 1100 FIBRO system are rinsed with Millipore 18 MΩ Water 3 times. The syringe is then loaded with Millipore 18 MΩ Water and any air bubbles are eliminated from the syringe before inserting into the DAT 1100 FIBRO system. The DAT 1100 FIBRO system is calibrated with the calibration standard provided by the manufacturer. The materials are handled with clean tweezers and cotton gloved hands to ensure minimum contact with the measured surface of the material. For each material tested, a total of at least 10 contact angle measurements are taken. The contact angle is reported as the average contact angle measured at 5 s for a material.

The following conditions are used for the DAT 1100 Fibro system: 1) Liquid is Millipore 18 MΩ Water; 2) Timeout is 0.2 minutes; 3) Number of Drops is 2-3 (per strip); 4) Drop size is 4 microliter; 5) Stroke pulse is 11; 6) Time collected is 0.10 s, 5 s and 10 s; 7) Steps is 1; 8) Minimum height is 8; 9) Minimum width is 10; 10) Capture Offset is 0; 11) Travel time is 2; 12) Pump delay is 5; 13) References Lines; 14) Mod threshold is 0; 15) Cannula Tip is 245; 16) Drop bottom is 97; and 17) Paper Position is 8, 18) Application Mode 1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A particle comprising a water-insoluble polymer and a volatile material produced by a PGSS process.

2. The particle according to claim 1 wherein the average particle size is less than 1 mm as measured according to the Particle Size Test Method described herein.

3. The particle according to claim 2 wherein the average particle size is less than 500 μm as measured according to the Particle Size Test Method described herein.

4. The particle according to claim 1 wherein the average particle size is greater than 1 nm as measured according to the Particle Size Test Method described herein.

5. The particle according to claim 1 wherein the particle exhibits a Morphology Coefficient F of greater than 0.2.

6. The particle according to claim 1 wherein the polymer comprises a copolymer of ethylene.

7. The particle according to claim 6 wherein the copolymer of ethylene comprises a copolymer of ethylene-vinyl acetate.

8. The particle according to claim 1 wherein the volatile material comprises a perfume.

9. The particle according to claim 1 wherein the volatile agent is at least partially encapsulated by the polymer.

10. The particle according to claim 1 wherein the particle further comprises a coating material.

11. The particle according to claim 1 wherein the particle further comprises a carrier particulate material to which the particle is attached.

12. A plurality of particles comprising a water-insoluble polymer and a volatile material produced by a PGSS process.

13. The plurality of particles according to claim 12 wherein greater than 80% of the plurality of particles exhibit a particle size of between 200 μm and 500 nm as measured according to the Particle Size Test Method described herein.

14. The plurality of particles according to claim 12 wherein the plurality of particles exhibit an average particle size distribution from about 250 μm to 100 nm as measured according to the Particle Size Test Method described herein.

15. A process for making a particle, the process comprising the step of depressurizing a solution comprising a water-insoluble polymer and a volatile material and a highly compressible fluid dissolved in the solution such that a particle comprising the water-insoluble polymer and the volatile material is produced.

16. The process according to claim 15 wherein the solution is produced by dissolving a highly compressible fluid in a solution comprising the polymer and the volatile material.

17. The process according to claim 15 wherein the process further comprises the step of coating the particle with a coating material.

18. The process according to claim 15 wherein the process further comprises the step of mixing a carrier with the particle.

19. A consumer product comprising a particle according to claim 1.

20. The consumer product according to claim 19 wherein the consumer product is selected from the group consisting of: shampoos, body washes, laundry detergents, dishwashing detergents, anhydrous liquid products, bar soaps, paper products, cosmetics, lotions, skin treating products, and mixtures thereof.
